(19) United States
(12) Patent Application Publication
(10) Pub. No.: US 2023/0040091 A1
(43) Pub. Date: Feb. 9, 2023

(54) SALIENT OBJECT DETECTION FOR ARTIFICIAL VISION

(71) Applicant: Commonwealth Scientific and Industrial Research Organisation, Acton (AU)

(72) Inventors: Nariman HABILI, Acton (AU); Jeremy OORLOFF, Acton (AU); Nick BARNES, Acton (AU)

(21) Appl. No.: 17/782,294

(22) PCT Filed: Nov. 30, 2020
(86) PCT No.: PCT/AU2020/051299

§ 371 Date: Jun. 3, 2022

(30) Foreign Application Priority Data
Dec. 5, 2019 (AU) 2019904611

(51) Int. Cl. A61F 9/08; A61N 1/36; G06T 7/11; G06T 7/194; G06T 7/149

(52) U.S. Cl. CPC A61F 9/08; A61N 1/3606; G06T 7/11; G06T 7/194; G06T 7/149; G06T 2207/20021

(57) ABSTRACT

There is provided a method for creating artificial vision with an implantable visual stimulation device. The method comprises receiving image data comprising, for each of multiple points of the image, a depth value, performing a local background enclosure calculation on the input image to determine salient object information, and generating a visual stimulus to visualise the salient object information using the visual stimulation device. Determining the salient object information is based on a spatial variance of at least one of the multiple points of the image in relation to a surface model that defines a surface in the input image.

### CROSS-REFERENCE TO RELATED APPLICATIONS

The present application claims priority from Australian Provisional Patent Application No 2019904611 filed on 5 Dec. 2019, the contents of which are incorporated herein by reference in their entirety.

### TECHNICAL FIELD

Aspects of this disclosure relate generally to the creation of artificial vision stimulus for use with an implantable visual stimulation device, and more specifically to systems and methods for optimising the efficacy of the same.

### BACKGROUND

Artificial vision systems which include implantable vision stimulation devices provide a means to provide vision information to a vision impaired user. An exemplary artificial vision system comprises an external data capture and processing component, and a visual prosthesis implanted in a vision impaired user, such that the visual prosthesis stimulates the user's visual cortex to produce artificial vision.

The external component includes an image processor, and a camera and other sensors configured to capture image of a field of view in front of a user. Other sensors may be configured to capture depth information, information relating to the field of view or information relating to the user. The image processor is configured to receive and convert this image information into electrical stimulation parameters, which are sent to a visual stimulation device implanted in the vision impaired user. The visual stimulation device has electrodes configured to stimulate the user's visual cortex, directly or indirectly, so that the user perceives an image comprised of flashes of light (phosphene phenomenon) which represent objects within the field of view.

A key component of visual interpretation is the ability to rapidly identify objects within a scene that stand out, or are salient, with respect to their surroundings. The resolution of the image provided to a vision impaired user via an artificial vision system is often limited by the resolution and colour range which can be reproduced on the user's visual cortex by the stimulation probes. As a result, important details may disappear as they are mapped to the same intensity value as their background. Accordingly, there is an emphasis on visually highlighting the objects, in the field of view, which are salient to the user.

Some fields of view contain multiple objects, or parts of objects, with a highly salient object located in front of less salient objects or surfaces. Accordingly, it is important for an artificial vision system to accurately determine the location and form of the highly salient objects, so that it may effectively present the saliency information to the user.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

According to one aspect of the disclosure, there is provided a method for creating artificial vision with an implantable visual stimulation device, the method comprising: receiving image data comprising, for each of multiple points of the image, a depth value; performing a local background enclosure calculation on the input image to determine salient object information; and generating a visual stimulus to visualise the salient object information using the visual stimulation device, wherein determining the salient object information is based on a spatial variance of at least one of the multiple points of the image in relation to a surface model that defines a surface in the input image.

The surface model may be a neighbourhood surface model which is spatially associated with the at least one of the multiple points of the image. The step of determining the salient object information may comprise determining a neighbourhood surface score for the at least one of the multiple points of the image, and wherein the neighbourhood surface score is based on a degree of the spatial variance of the at least one of the multiple points of the image from the neighbourhood surface model.

The local background enclosure calculation may comprise calculating a local background enclosure result for the at least one of the multiple points of the image.

The method for creating artificial vision with an implantable visual stimulation device may further comprise adjusting the local background enclosure result based on the neighbourhood surface score, and adjusting the local background enclosure result comprises reducing the local background enclosure result based on the degree of spatial variance.

The neighbourhood surface model may be representative of a virtual surface defined by a plurality of points of the image in the neighbourhood of the at least one of the multiple points of the image. The neighbourhood surface model may be a planar or non-planar surface model.

The image data may be spatially segmented into a plurality of superpixels, wherein each superpixel comprises one or more pixels of the image. At least one of the multiple points of the image may be contained in a selected superpixel of the plurality of superpixels. In one example, the neighbourhood comprises a plurality of neighbouring superpixels located adjacent to the selected superpixel. In another example, the neighbourhood comprises a plurality of neighbouring superpixels located within a radius around the selected superpixel. In yet another example, the neighbourhood comprises the entire image.

A random sample consensus method may be used to calculate the neighbourhood surface model for the target superpixel, based on a three dimensional location of the superpixels within the neighbourhood of the target superpixel.

The method for creating artificial vision with an implantable visual stimulation device may further comprises performing post-processing of the salient object information, and the post-processing comprises performing one or more of depth attenuation, saturation suppression and flicker reduction.

According to another aspect of the disclosure, there is provided an artificial vision device for creating artificial vision, the artificial vision device comprising an image processor configured to: receive image data comprising, for each of multiple points of the image, a depth value; perform a local background enclosure calculation on the input image to determine salient object information; and generate a visual stimulus to visualise the salient object information using the visual stimulation device, wherein determining the salient object information is based on a spatial variance of at least one of the multiple points of the image in relation to a surface model that defines a surface in the input image.

### BRIEF DESCRIPTION OF DRAWINGS

Examples will now be described with reference to the following drawings, in which:

FIG. 1a-1c illustrate an example image and the calculation of local background enclosure results for select regions of the image;

FIG. 2 is block diagram illustrating an artificial vision system comprising an image processor in communication with a visual stimulation device;

FIG. 3 is a flowchart illustrating a method, as performed by an image processor, of generating visual stimulus;

FIG. 4 is a flowchart illustrating a method, as performed by an image processor, of receiving image data;

FIG. 5a illustrates the segmentation of the image of FIG. 1a into a plurality of superpixels;

FIG. 5b illustrates the LBE adjustment for surfaces of the image of FIG. 1a;

FIG. 6 is a flowchart illustrating a method, as performed by an image processor, of calculating local background enclosure results;

FIG. 7 is a flowchart illustrating a method, as performed by an image processor, of calculating a neighbourhood surface score;

FIG. 8 is a flowchart illustrating a method, as performed by an image processor, of determining a surface model;

FIG. 9 is a flowchart illustrating a method, as performed by an image processor, of post processing phosphene values.

### DESCRIPTION OF EMBODIMENTS

This disclosure relates to image data including a depth channel, such as from a laser range finder, ultrasound, radar, binocular/stereoscopic images or other sources of depth information.

An artificial vision device can determine the saliency of an object within a field of view, represented by an image of a scene including a depth channel by measuring the depth contrast between the object and its neighbours (i.e. local scale depth contrast) and the depth contrast between the object and the rest of the image (i.e. global scale depth contrast).

Salient objects within a field of view tend to be characterised by being locally in front of surrounding regions, and the distance between an object and the background is not as important as the observation that the background surrounds the object for a large proportion of its boundary. The existence of background behind an object, over a large spread of angular directions around the object indicates pop-out structure of the object and thus implies high saliency of the object. Conversely, background regions in the field of view are less likely to exhibit pop-out structures, and may be considered to be less salient.

A technique for determining the saliency of an object in a field of view, based on these principles, is the calculation of a local background enclosure for candidate regions within an image of the field of view. Such a method has been described in “Local background enclosure for RGB-D salient object detection” (Feng D, Barnes N, You S, et al., Proceedings of the IEEE Conference on Computer Vision and Pattern Recognition, 2016, 2343-2350) [1], which is incorporated herein by reference.

The Local Background Enclosure (LBE) technique measures saliency within an image based on depth information corresponding to pixels of the image. Specifically, the LBE technique analyses an object and more particularly, a candidate region that is a part of that object. A candidate region can be a single pixel or multiple pixels together in a regular or irregular shape.

The LBE technique defines a local neighbourhood around a candidate region and determines the spread and size of angular segments of pixels within that local neighbourhood (such as pixels within a predefined distance) that contain background, noting that the background is defined with respect to the candidate region. That is, a first object in front of a background may be part of the background of a second object that is in front of the first object.

An LBE calculation incorporates at least two components. The first, which is broadly proportional to saliency, is an angular density of background around the region. This encodes the intuition that a salient object is in front of most of its surroundings. The second LBE component, which is broadly inversely proportional to saliency, is the size of the largest angular region containing only foreground, since a large value implies significant foreground structure surrounding the object.

The LBE technique provides a method of determining the location and form of salient objects within a scene; however, there may be situations in which the scene includes a surface which is not highly salient to the user, but for which the LBE technique indicates high saliency due to the way the LBE technique calculates saliency.

One such situation occurs when an object of high saliency is located in front of a surface of less saliency, and the surface of less saliency is located in front of the background. In this situation, it is desirable to distinguish the location and form of the less salient surface from the location and form of the highly salient object within the scene. A technique for achieving this distinction is to suppress the visual representation of the less salient surface within the artificial vision stimulus.

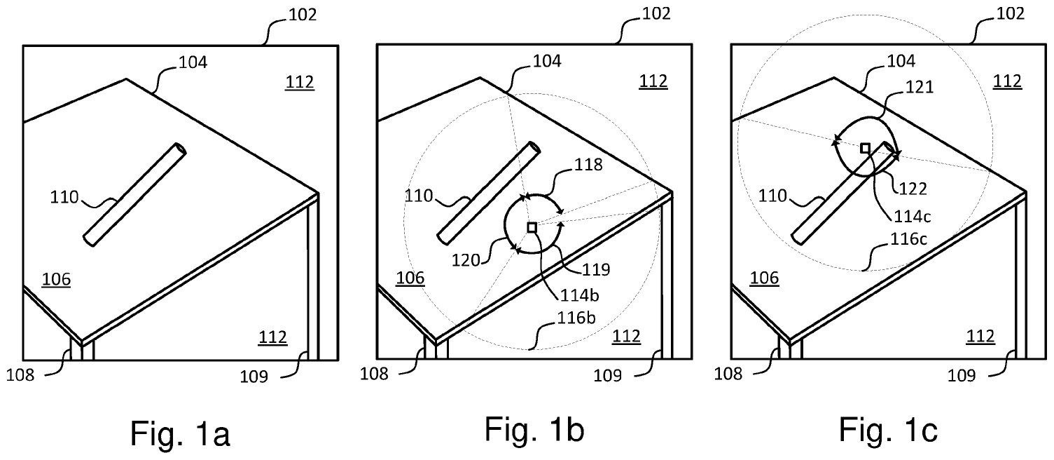

FIGS. 1a-1c illustrate an example in which it is desirable to suppress the saliency representation for a surface of less saliency. FIGS. 1a-1c illustrate schematic image 102 captured by a camera with a depth sensor. The image 102 is shown in monochrome, and omits natural texture, luminance and colour, for the purposes of clear illustration. The image 102 depicts a table 104 as viewed from a view point above and in front of the table 104. The table comprises a flat table surface 106, and table legs 108, 109. On the table surface 106 is an object 110. In this example, the object 110 is a pen; however it is to be understood that the object may be another salient object located on or in front of the table surface. Behind the table 104 is the background, such as a distant surfaces 112, which may be the floor under the table 104.

In image 102, the pen 110 is the highly salient object, and it is desirable that the location and form of the object 110 is highlighted to the user, via artificial vision stimulus.

An artificial vision device may perform a series of LBE calculations on candidate regions of image 102 to determine the location and form of salient objects. An exemplary LBE calculation to determine salient objects within a scene takes into consideration the angular density of background around a candidate region, and the size of the largest angular region containing only foreground.

FIG. 1b illustrates image 102 with overlaid information to illustrate an example calculation of LBE for candidate region 114b. Candidate region 114b is shown in this example as a square shaped region, although it is to be understood that the shape and size of a candidate region may be a single pixel, or set of pixels in a regular or irregular shape (also referred to as “superpixel”).

Region 114b represents a section of table surface 106. A local neighbourhood around candidate region 114b is determined based on a fixed radius r, and is illustrated by dashed line 116b. The local neighbourhood encompasses sections of the image 102 representing the table surface 106, the object 110 and the distance surfaces 112.

Within the local neighbourhood 116b, candidate region 114b has two large angular segments 118, 119 which both include parts of background 112. This means that the LBE angular density score, which measures the angular density of background around a candidate region 114b, will be a moderately high number. Additionally, within the local neighbourhood, candidate region 114b has a large angular segment 120 which includes only table surface 106. This means that the angular gap score, which measures the size of the largest angular region containing only foreground, will be moderate. Accordingly, the LBE calculations for candidate region 114b will result in a high LBE score, and the candidate region will be represented as a salient region to the user, via artificial vision stimulus.

FIG. 1c illustrates image 102 with overlaid information to illustrate an example calculation of LBE for another candidate region 114c. Again, candidate region 114c is shown as a square shaped region, although it is to be understood that the shape and size of a candidate region is not limited to such.

Region 114c is a section of table surface 106. A local neighbourhood around candidate region 114c is illustrated by dashed line 116c. The local neighbourhood encompasses sections of the image representing the table surface 106, object 110 and background 112.

Within the local neighbourhood, candidate region 114c has a large angular segment 121, of approximately 180 degrees, which include parts of background 112. This means that the angular density score for target region 114c, will be high. Additionally, within the local neighbourhood, candidate region 114c has a large angular segment 122, of approximately 180 degrees, which includes only table surface 106. This means that the angular gap score, will be moderate. Accordingly, the LBE calculations for candidate region 114c will result in a moderately high LBE score, and the candidate region 114c will be represented as a moderately salient region to the user, via artificial vision stimulus.

As described in relation to FIGS. 1b and 1c, LBE calculations comprising only a consideration of angular density score and angular gap score may result in regions around the salient object 110 being considered to have a moderate or high saliency. This may result in the user not being able to clearly distinguish the form of a highly salient object located in front of a less salient object, from the artificial visual stimulus.

In another example, if the scene incorporates a surface, such as a wall, floor or table top, which is located in front of a background, the LBE algorithm may consider this surface to be salient. Accordingly, the surface will be visually highlighted to the user as higher intensity luminance in the artificial visual stimulus. However, if a salient object is located in the scene, the highlighting of the regions representing the surface may result in the salient object not being visually distinguished from the surface, and therefore not appearing salient to the user.

Accordingly, it is desirable to suppress the saliency of a surface within a scene in order to highlight the location and form of highly salient objects within the scene. It is noted that the surface may be a plane, such as a wall or floor, a regular surface, such as a curve wall or spherical surface, or an irregular surface.

The present disclosure describes the incorporation of a neighbourhood surface score which adjusts the LBE result for regions of an image in which an artificial vision system determines that the region is part of a surface within the scene. The method by which the artificial vision system calculates and applies the neighbour surface score will be described in relation to the following examples.

### Artificial Vision Device

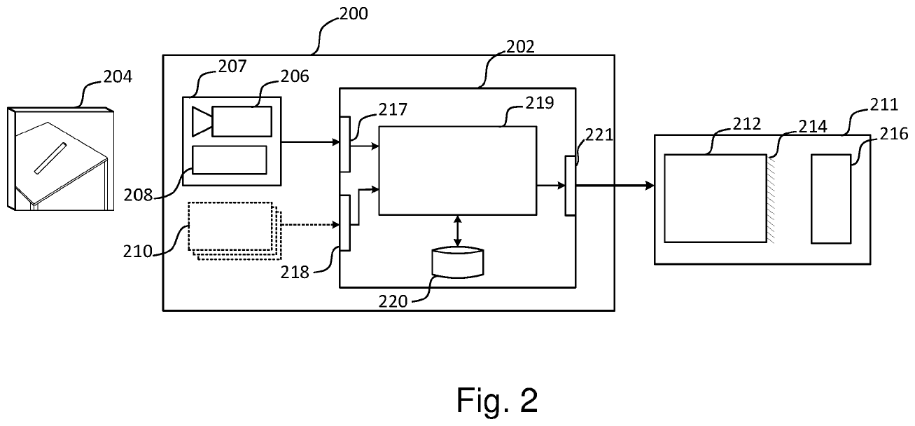

FIG. 2 is a block diagram illustrating an exemplary structure of an artificial vision device 200 which is configured to generate a visual stimulus, representative of a scene 204, for a vision impaired user 211. In particular, the artificial vision device 200 is configured to generate a representation of object saliency, for objects within the scene 204, for the vision impaired user. The scene 204 represents the physical environment of the user and is naturally three dimensional.

The vision impaired user 211 has an implanted visual stimulation device 212 which stimulates the user's visual cortex 216, either directly or indirectly, via electrodes 214 to produce artificial vision.

The artificial vision device may comprise a microprocessor based device, configured to be worn on the person of the user. The artificial vision device 200 illustrated in FIG. 2, includes an image sensor 206, a depth sensor 208 and an image processor 202. In other embodiments the image and depth sensors may be located external to the artificial vision device 200.

The aim is to enable the vision-impaired user to perceive salient objects within the view of the image sensor 206. In particular, the aim is to generate a stimulation signal, such that the user perceives salient objects as highlighted structures. For example, the user may, as a result of the stimulation, perceive salient objects as white image structures and background as black image structures or vice versa. This may be considered similar to ‘seeing’ a low resolution image. While the resolution is low, the aim is to enable the vision-impaired user to navigate everyday scenarios with the help of the disclosed artificial vision system by providing salient objects in sufficient detail and frame rate for that navigation and the avoidance of immediate dangers.

The image processor 202 receives input data representing multiple points (i.e. pixels) of the scene 204 from an image sensor 206 (such as an RGB camera), and a depth sensor 208 (such as a laser range finder). The image sensor 206 may be a high resolution digital camera which captures luminance information representing the field of view of the scene 204 from the camera's lens, to provide a two-dimensional pixel representation of the scene, with brightness values for each pixel. The image sensor 206 may be configured to provide the two-dimensional representation of the scene in the form of greyscale image or colour image.

The depth sensor 208 captures a representation of the distance of points in the scene 204 from the depth sensor. The depth sensor provides this depth representation in the form of a depth map which indicates a distance measurement for each pixel in the image. The depth map may be created by computing stereo disparities between two space-separated parallel cameras. In another example, the depth sensor is a laser range finder that determines the distance of points in the scene 204 from the sensor by measuring the time of flight and multiplying the measured time of flight by the speed of light and divide by two to calculate a distance. In other examples, the pixels of the depth map represent the time of flight directly noting that a transformation that is identical for all pixels should not affect the disclosed method, which relies on relative differences in depth and not absolute values of the distance.

The image sensor 206 and the depth sensor 208 may be separate devices. Alternatively, they may be a single device 207, configured to provide the image and depth representations as separate representations, or to combine the image and depth representations into a combined representation, such as an RGB-D representation. An RGB-D representation is a combination of an RGB image and its corresponding depth image. A depth image is an image channel in which each pixel value represents the distance between the image plane and the corresponding point on surface within the RGB image. So, when reference is made herein to an ‘image’, this may refer to a depth map without RGB components since the depth map essentially provides a pixel value (i.e. distance) for each pixel location. In other words, bright pixels in the image represent close points of the scene and dark pixels in the image represent distant points of the scene (or vice versa).

For simplicity, the image sensor 206 and the depth sensor 208 will be described herein as a single device which is configured to capture an image in RGB-D. Other alternatives to image capture may, of course, also be used.

In other embodiments, the image processor 202 may receive additional input from one or more additional sensors 210. The additional sensors 210 may be configured to provide information regarding the scene 204, such as contextual information regarding salient objects within the scene 204 or categorisation information indicating the location of the scene 204. Alternatively or additionally, the sensors 210 may be configured to provide information regarding the scene 204 in relation to the user, such as motion and acceleration measurements. Sensors 210 may also include eye tracking sensors which provide an indication of where the user's visual attention is focused.

The image processor 202 processes input image and depth information and generates visual stimulus in the form of an output representation of the scene 204. The output representation is communicated to a visual stimulation device 212, implanted in the user 211, which stimulates the user's visual cortex 216 via electrodes 214.

The output representation of the scene 204 may take the form, for example, of an array of values which are configured to correspond with phosphenes to be generated by electrical stimulation of the visual pathway of a user, via electrodes 214 of the implanted visual stimulation device 212. The implanted visual stimulation device 212 drives the electrical stimulation of the electrodes in accordance with the output representation of the scene 204, as provided by the image processor 202.

The output data port 221 is connected to an implanted visual stimulation device 212 comprising stimulation electrodes 214 arranged as an electrode array. The stimulation electrodes stimulate the visual cortex 216 of a vision impaired user. Typically, the number of electrodes 214 is significantly lower than the number of pixels of camera 206. As a result, each stimulation electrode covers an area of the scene 204 captured by multiple pixels the sensors 207.

Typically, electrode arrays 214 are limited in their spatial resolution, such as 8×8, and in their dynamic range, that is, number of intensity values, such as 3 bit resulting in 8 different values; however, the image sensor 206 can capture high resolution image data, such as 640×480 with 8 bit.

Often, the image processor 202 is configured to be worn by the user. Accordingly, the image processor may be a low-power, battery-operated unit, having a relatively simple hardware architecture.

In an example, as illustrated in FIG. 2, the image processor 202 includes a microprocessor 219, which is in communication with the image sensor 206 and depth sensor 208 via input 217, and is communication with other sensors 210 via input 218. The microprocessor 219 is operatively associated with an output interface 221, via which image processor 202 can output the representation of the scene 204 to the visual stimulation device 212.

It is to be understood that any kind of data port may be used to receive data on input ports 217 and 218 and to send data on output port 221, such as a network connection, a memory interface, a pin of the chip package of processor 219, or logical ports, such as IP sockets or parameters of functions stored in memory 220 and executed by processor 219.

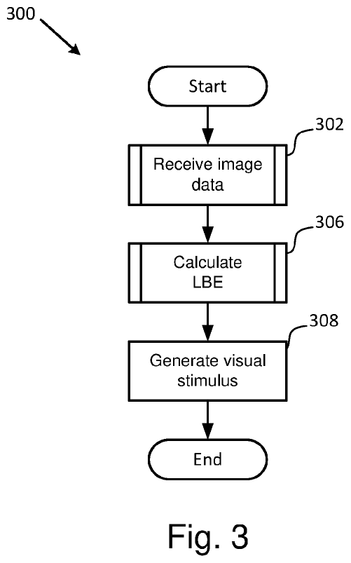

The microprocessor 219 is further associated with memory storage 220, which may take the form of random access memory, read only memory, and/or other forms of volatile and non-volatile storage forms. The memory 220 comprises, in use, a body of stored program instructions that are executable by the microprocessor 219, and are adapted such that the image processor 202 is configured to perform various processing functions, and to implement various algorithms, such as are described below, and particularly with reference to FIGS. 3 to 9.

The microprocessor 219 may receive data, such as image data, from memory storage 220 as well as from the input port 217. In one example, the microprocessor 219 receives and processes the images in real time. This means that the microprocessor 219 performs image processing to identify salient objects every time a new image is received from the sensors 207 and completes this calculation before the sensors 207 send the next image, such as the next frame of a video stream.

It is to be understood that, in other embodiments, the image processor 202 may be implemented via software executing a general-purpose computer, such as a laptop or desktop computer, or an application specific integrated device or a field programmable gate array. Accordingly the absence of additional hardware details in FIG. 1 should not be taken to indicate that other standard components may not be included within a practical embodiment of the invention.

### Method for Creating Artificial Vision

FIG. 3 illustrates a method 300 performed by the image processor 202, for creating artificial vision with an implantable visual stimulation device 212. Method 300 may be implemented in software stored in memory 220 and executed on microprocessor 219. Method 300 is configured through the setting of configuration parameters, which are stored in memory storage 220.

In step 302, the image processor 202 receives image data from the RGB_D camera 207. The image data comprises an RGB image, of dimensions x by y pixels, and a corresponding depth channel. In one example, the image data only comprises the depth channel.

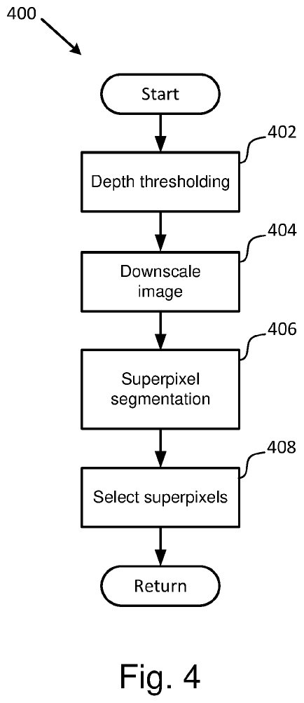

The image processor 202 pre-processes the received image data to prepare the data for subsequent processing. Method 400 in FIG. 4 illustrates the steps of pre-processing the received image data. In step 402, image processor 202 applies threshold masks to the depth image to ensure the pixels of the depth image are each within the defined acceptable depth range. The acceptable depth range for performing visual stimulation processing may be defined through configuration parameters which represent a maximum depth threshold and a minimum depth threshold. The depth threshold configuration parameters may vary in accordance with the type of scene being viewed, contextual information or the preferences of the user. The depth image may also be smoothed to reduce spatial or temporal noise. It is noted here that some or all configuration parameters may be adjusted either before the device is implanted or after implantation by a clinician, a technician or even the user itself to find the most preferable setting for the user.

In step 404, the image provided by image sensor 206 may be modified to reduce the spatial resolution of an image, and hence to reduce the number of pixels to be subsequently processed. The image may be scaled in the horizontal and vertical dimensions, in accordance with configuration parameters stored in the image processor.

In one example, image data of a reduced spatial resolution is determined by selecting every second pixel of the higher resolution image data. As a result, the reduced spatial resolution is half the high resolution. In other examples, other methods for resolution scaling may be applied.

In step 406, the image processor segments the RGB-D image, represented by pixel grid I(x,y). For computational efficiency and to reduce noise from the depth image, instead of directly working on pixels, the image processor segments the input RGB-D image into a set of superpixels according to their RGB value. In other examples, the image processor segments the input image data into a set of superpixels according to their depth values. This means, the input image does not necessarily have to include colour (RGB) or other visual components but could be purely a depth image. Other ways of segmentation may equally be used. In other words, image segmentation is the process of assigning a label (superpixel ID) to every pixel in an image such that pixels with the same label share certain characteristics (and belong to the same superpixel).

A superpixel is a group of spatially adjacent pixels which share a common characteristic (like pixel intensity, or depth). Superpixels can facilitate artificial vision algorithms because pixels belonging to a given superpixel share similar visual properties. Furthermore, superpixels provide a convenient and compact representation of images that can facilitate fast computation of computationally demanding problems.

### SLIC Superpixel Segmentation

In the example of FIG. 4, the image processor 202 utilises the Simple Linear Iterative Clustering (SLIC) [2] algorithm to perform segmentation; however, it is noted that other segmentation algorithms may be applied. The SLIC segmentation algorithm may be applied through use of the OpenCV image processing library. The SLIC segmentation process is configured through the setting of configuration parameters, including a superpixel size parameter which determines the superpixel size of the returned segment, and a compactness parameter which determines the compactness of the superpixels within the image.

The processing power required to perform SLIC segmentation depends on the resolution of the image, and the number of pixels to be processed by the segmentation algorithm. The resolution scaling step 404 assists with reducing the processing requirements of step 406 by reducing the number of pixels required to be processed by the segmentation algorithm.

### Segmentation Example

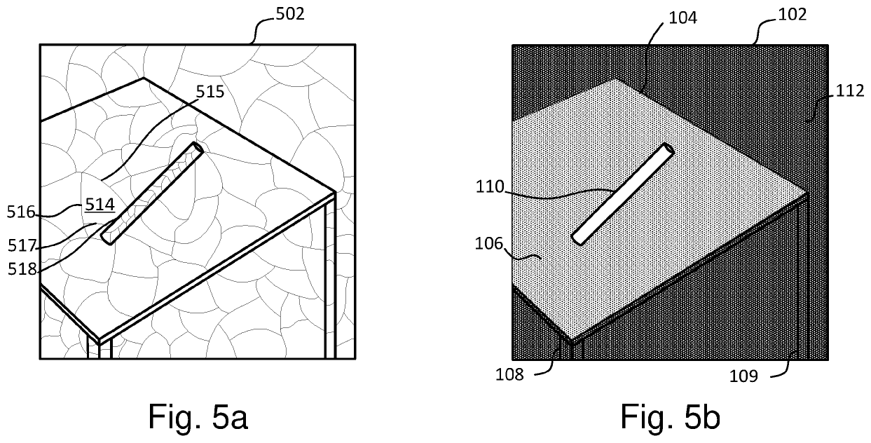

FIG. 5 illustrates a representation 502 of the image 102 of FIG. 1a, showing the segmentation of image 102 into a plurality of superpixels as a result of performing the superpixel segmentation step 406 over the image 102. Each superpixel shown in FIG. 5 contains one or more adjacent pixels of the image. The superpixels are bounded by segmentation lines. For example, superpixel 514, which includes pixels illustrating table surface 106, is bounded by segmentation lines 515, 516, 517 and 518. Segmentation line 518 is collocated with an edge of the object 110.

It can be seen that the superpixels are of irregular shape and non-uniform size. In particular, superpixels representing the distant surface 112 are spatially larger, encompassing more pixels, than the superpixels representing the table surface 106. Furthermore, the superpixels of the object 110 are spatially smaller and encompass few pixels. This is indicative of the example object 110 having varying texture, luminance or chrominance.

Image processor 202 uses the superpixels determined in segmentation step 406 within Local Background Enclosure calculations to identify the presence and form of salient objects.

### Superpixel Selection

In one example, to reduce computational complexity, the image processor 202 performs LBE calculations on only a select subset of the superpixels determined in segmentation step 406 In accordance with such an example, the image processor 202 performs superpixel selection in step 3408, prior to performing the LBE calculations in step 306. The image processor may select the subset of superpixels to perform LBE on based upon whether the superpixel is collocated with a phosphene location in an array of phosphene locations, and/or whether the depth of the corresponding superpixel is within a configured object depth threshold.

The object depth threshold indicates the distance from the depth sensor 208 at which an object may be considered to be salient by the image processor. The object depth threshold may comprise a maximum object depth threshold and a minimum object depth threshold. The maximum distance at which an object would be considered not salient may depends upon on the context of the 3D spatial field being viewed by the sensors. For example, if the field of view is an interior room, objects that are over 5 meters away may not be considered to be salient objects to the user. In contrast, if the field of view is outdoors, the maximum depth at which objects may be considered salient may be significantly further.

If the depth of the superpixel corresponding with a phosphene location is not within the defined object depth threshold, the superpixel may not be selected by the image processor for subsequent LBE calculation.

In yet another example, the image processor has access to an object model for the scene 204, which comprises information representing the location and form of one or more predetermined objects within the field of view. The location and form of the predetermined objects may be determined by the image processor. Alternatively, the object model may be provided to the image processor. In this example, the image processor appends the object model information, which represents the location and form of one or more predetermined objects, to the salient object information after LBE calculations have been performed, thus reducing the number of LBE calculations that are performed for a particular image frame.

For some embodiments, or some situations, it may be desirable or feasible to calculate the LBE for every superpixel within the image. In this case, a configuration parameter may be set to indicate that the superpixel selection process may be omitted, and the output of step 304 will be a list of every superpixel in the image.

### Calculate LBE

In step 306, the image processor 202 calculates the local background enclosure (LBE) result for each of the superpixels in the list of selected superpixels provided by step 304.

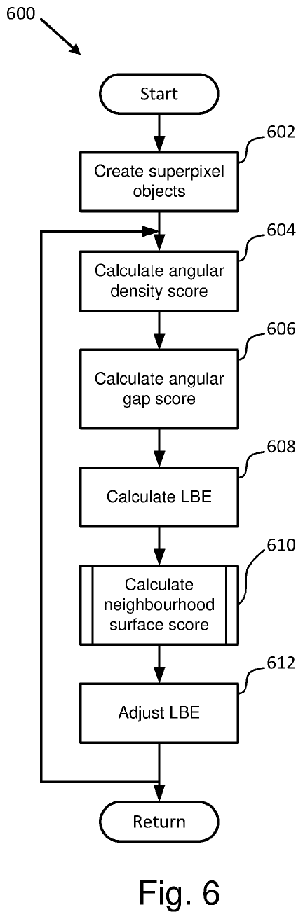

FIG. 6 illustrates the steps taken by the image processor 202 to calculate the LBE result for the list of selected superpixels. In step 602, the image processor 202 creates superpixel objects for each superpixel in the list of selected superpixels. In one example, the image processor creates the superpixel objects by calculating the centroid of each superpixel, and the average depth of the pixels in each superpixel. When calculating the average depth, the image processor may ignore depth values equal to zero.

For each of the selected superpixels, the image processor additionally calculates the standard deviation of the depth, and a superpixel neighbourhood comprised of superpixels that are within a defined radius of the superpixel.

In steps 604 to 608, for each selected superpixel P, the image processor 102 calculates, based on superpixel's neighbourhood, an angular density score F, an angular gap score G, and a neighbourhood plane score for each superpixel. These scores are combined to produce the LBE result S, for the superpixel.

### Angular Density Score

In step 604, the image processor calculates the angular density of the regions surrounding P with greater depth than P, referred to as the local background. A local neighbourhood N_{P} of P, consisting of all superpixels within radius r of P. That is, N_{P}={Q|Pc_{P}−c_{Q}P₂<r}, where c_{P} and c_{Q} are superpixel centroids.

The local background B (P,t) of P is defined as the union of all superpixels within a neighbourhood N_{P} that have a mean depth above a threshold t from P.

B(P,t)=∪{P′∈N_{P}|D(P′)>D(P)+t}, (1)

where D(P) denotes the mean depth of pixels in P.

Method 600 defines a function ƒ(P,B(P,t)) that computes the normalised ratio of the degree to which B (P,t) encloses P.

$$f ( P , B ( P , t ) ) = \frac{1}{2 π} {\int }_{0}^{2 π} I ( θ , P , B ( P , t ) ) d θ , \qquad ( 2 )$$

where I(θ,P,B(P,t))) is an indicator function that equals 1 if the line passing through the centroid of superpixel p with angle θ intersects B(P,t), and 0 otherwise.

Thus ƒ(P,B(P,t)) computes the angular density of the background directions. Note that the threshold t for background is an undetermined function. In order to address this, as frequently used in probability theory, we employ the distribution function, denoted as F(P), instead of the density function ƒ, to give a more robust measure. We define F(P) as:

F(P)=∫₀^{σ}ƒ(P,B(P,t))dt, (3)

where σ is the standard deviation of the mean superpixel depths within the local neighbourhood of P.

This is given by

$${σ}^{2} = \frac{1}{\vert B ( P , 0 ) \vert} {\sum }_{Q ∈ B ( P , 0 )} {( D ( Q ) - \overline{D} )}^{2} ,$$

where

$$\overline{D} = \frac{1}{\vert B ( P , 0 ) \vert} {\sum }_{Q ∈ B ( P , 0 )} D ( Q ) .$$

This implicitly incorporates information about the distribution of depth differences between P and its local background.

### Angular Gap Score

In step 606, the image processor 202 calculates an angular gap score G(P). The angular gap score provides an adjustment in the situation where two superpixels have similar angular densities; however, one of the two superpixels appears to have higher saliency due to background directions which are more spread out. To provide this adjustment, the method 600 applies the function g (P,Q) to find the largest angular gap of Q around P and incorporate this into the saliency score.

$$g ( P , Q ) = \frac{1}{2 π} \cdot {max}_{( {θ}_{1} , {θ}_{2} ) ∈ Θ} \{ \vert {θ}_{1} - {θ}_{2} \vert \} , \qquad ( 4 )$$

where Θ denotes the set of boundaries (θ₁,θ₂) of angular regions that do not contain background:

Θ={(θ₁,θ₂)|I(θ,P,Q)=0 ∀θ∈[θ₁,θ₂]}. (5)

The angular gap statistic is defined as the distribution function of 1−g:

G(P)=∫₀^{σ}1−g(P,B(P,t))dt. (6)

The LBE result is given by:

S(P)=F(P)·G(P). (7)

In step 608, the image processor 202 combines the angular density score, the angular gap score to give the LBE result for a superpixel. In one example, the scores are combined through an unweighted multiplication. In other examples, weighted or conditional multiplication methods may be used to combine the scores to produce an LBE result for a superpixel.

### Neighbourhood Surface Score

In step 610, the image processor calculates a third score, namely, a neighbourhood surface score, for each of the selected superpixels to provide an adjustment to the LBE result in the situation where the superpixel represents part of a less salient surface.

To calculate the neighbourhood surface score, the image processor 102 determines a neighbourhood surface model which defines the location and form of a virtual surface in the neighbourhood of the superpixel. The neighbourhood surface score for a superpixel is based on a spatial variance of the superpixel from the neighbourhood surface model. If there is a high degree of spatial variance of a superpixel from the neighbourhood surface model, the image processor provides a neighbourhood surface score which preserves the LBE result for the superpixel. Conversely, if there is a low degree of spatial variance of a superpixel from the neighbourhood surface model, the image processor provides a neighbourhood surface score which somewhat suppresses the LBE result for the superpixel.

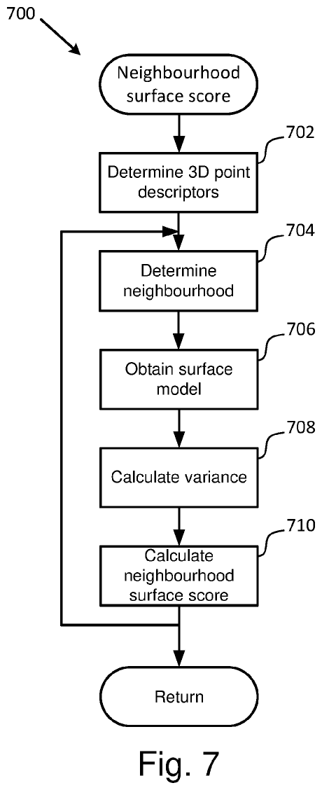

An example method by which the image processor 202 calculates a neighbourhood surface score is illustrated by method 700 in FIG. 7. Method 700 is configured to calculate the neighbourhood surface score for each superpixel in the list of selected superpixels, as determined in step 408. It is noted, however, that the image processor may be configured to calculate a neighbourhood surface score for a group of superpixels, individual pixels or groups of pixels.

### Determine 3D Descriptors

In step 702, the image processor 202 calculates, for each superpixel in the image 502, a three-dimensional (3D) point descriptor, based on each superpixel's position with regard to the x dimension of the image 502, they dimension of the image 502, and the depth dimension z, which is based on depth measurements. The 3D point descriptor may be in the form of a numerical tuple, calculated and stored for each superpixel. In one example, the 3D point descriptor is the centroid of the superpixel, as calculated in step 602. In another example, the 3D point descriptor may represent the mid-point of the superpixel's range in each of the three dimensions. In one example, points are transformed using the intrinsic camera properties. The reason for this is: If a camera is pointed at a flat wall in front of the camera and processor 202 extracts distances along a horizontal line perpendicular to the camera axis, the values may be (5, 4.5, 4(middle), 4.5, 5) because the camera is not the same distance from each point. This will make a surface reconstructed using raw depth data appear curved when it is actually flat. Transforming the data accounts for this issue. Therefore, processor 202 may transform the input data on the basis of the camera properties to obtain accurate 3D points.

The image processor iterates through steps 704 to 710 for each of the selected superpixels.

### Determine Neighbourhood

In step 704, the image processor 202 determines a neighbourhood around a target superpixel 514. In one example, the neighbourhood comprises superpixels which are immediately adjacent to the target superpixel. In another example, the neighbourhood comprises superpixels which are located within a set or configurable radius of the target superpixel, as determined in step 602. With regard to the example illustrated in FIG. 5, the neighbourhood is configured to comprise the entire image 502.

### Obtaining Surface Model

The image processor 202 then obtains 704 a surface model associated with the determined neighbourhood. A surface model defines a virtual surface which approximates the form and location of a surface within a nominated region of a scene. In one example, the surface model is a mathematical representation of a virtual surface, defined based on the x, y and depth dimensions of an image.

The surface modelled by the surface model may be the predominant surface within a scene, such that it is a surface upon which a majority number of points of the image are located, or a closely located. With reference to the example scene illustrated in FIG. 1, the predominant surface is the top surface 106 of the table 104. A neighbourhood surface model, for which the neighbourhood has been defined as the entire image 102, describes the form and location of the table top surface 106.

In another example, the scene may comprise a framed picture hanging on a wall. The predominant surface in that situation may be the wall. A surface model may be defined for the entirety of the scene, or for a part of scene, depending on the determined neighbourhood. Accordingly, there may be a plurality of surface models associated with a single image. A surface model may be indicative of a planar surface or a non-planar surface.

In accordance with one example, the image processor 202 obtains the surface model from memory 220. In a further example, the image processor obtains the surface model from an external source. In accordance with the example illustrated in FIGS. 5 to 8, the image processor 202 calculates a planar surface model based on the 3D point descriptors determined for a neighbourhood associated with a target superpixel 514.

### Determining a Planar Surface Model

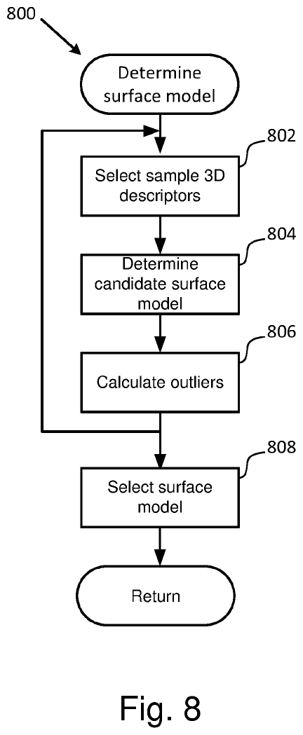

FIG. 8 illustrates an example method, as performed by the image processor 202, for calculating a surface model for a neighbourhood associated with a target superpixel. The neighbourhood for this example comprises the entire image 102, although in other examples the neighbourhood may comprise a sub-set of an image.

In this example, the image processor 202 utilises a random sample consensus (RANSAC) method to calculate a suitable surface model for the neighbourhood associated with a target superpixel. In broad terms, a RANSAC method is an iterative method used to estimate parameters of a mathematical model from a set of observed data that contains outliers. In the context of the embodiment illustrated by method 800, the RANSAC method may be applied through application of the following steps.

In step 802, the image processor 202 selects a plurality of sample 3D point descriptors associated with superpixels within the neighbourhood of target superpixel. In one example, the image processor selects the 3D descriptors pseudo-randomly. In another example, the image processor selects 3D descriptors which are spaced approximately equidistance from the target superpixel.

If the image processor seeks to determine a planar surface model, three 3D point descriptors are selected. The three 3D point descriptors define a candidate planar surface in the virtual space defined by the x dimension of the image, they dimension of the image, and the depth dimension z. The image processor mathematically defines the candidate planar surface, with respect to the dimensions of the image 502.

If the image processor seeks to determine a non-planar surface model, the image processor may be configured to select more than three 3D descriptors. For example, for a polynomial surface model, processor may select at least the number of descriptors as there are unknowns in the model. The processor may perform a least squares optimisation to fit the model to the descriptors. Other surface models may equally be used, such as wavelet or spline models. Further, non-mathematical or non-analytical approaches may be used, such as statistical models or models defined by empirical data collection, such as classification using a trained machine learning model.

In step 806, the image processor determines whether the candidate surface model is a suitable surface model to represent the neighbourhood, by considering the superpixels within the neighbourhood which are outliers from the candidate surface.

The degree at which a superpixel may be spatially variant from the candidate surface before being considered an outlier to the surface may be configurable through the setting of configuration parameters. In one example, a surface variance configuration parameter is defined which defines an acceptable spatial variance ratio. For example, if a 3D point descriptor of a superpixel lies at a distance which is more than the surface variance multiplied by the superpixel's depth away from the candidate surface, the superpixel is considered to be an outlier.

The image processor repeats the outlier determination for each of the remaining superpixels within the neighbourhood, taking note of the number of superpixels which are determined to be outliers to the candidate surface model, and the spatial variance (i.e. distance) of each of the superpixels from the candidate surface.

The image processor repeats the steps of selecting sample 3D descriptors 802, determining a candidate surface model 804, and calculating outliers 806 to the candidate surface model multiple times. In one example, the number of iterations of steps 802 to 806 may be preconfigured. In another example, at the completion of each iteration of steps 802 to 806, the image processor 202 may determine whether further iterations are required, based on the number of outliers, or the spatial variance of outliers.

At the completion of the iteration of steps 802 to 806, the image processor 202 determines which of the plurality of candidate surface models, determined in step 804, was the best fit for the neighbourhood of the target superpixel. To determine the best fit, the image processor may consider the number of superpixels which were considered to be outliers in step 806, and/or the delta distances of the superpixels from the candidate surface model. In one example, the best fit is considered to be the candidate surface model with the least number of superpixels that were considered to be outliers. In another example, the best fit surface model is the candidate surface model in which the sum of the delta distances of all the outlying superpixels is the least.

Once the image processor has selected 808 the best fit candidate surface model, the best fit candidate surface model, the image processor obtains that neighbourhood surface model for the target region, as illustrated by step 706.

### Calculating Variance

In step 708, the image processor 202 calculates the spatial variance of the target superpixel's 3D point descriptor from the obtained neighbourhood surface model.

A target superpixel having high variance from the surface model indicates that the superpixel is not aligned with the neighbouring surface, and that the superpixel represents a region that is set apart from its neighbouring surface. Accordingly, the region represented by the target superpixel may be part of a salient object. In this case, it is desirable to preserve the LBE result so that the salient object is highlighted to the user.

Conversely, a target superpixel having a low variance from the surface model indicates that a superpixel is aligned with the neighbouring surface, and that the superpixel may represent a section of the neighbouring surface. Accordingly, the region represented by the superpixel may not be part of a salient object. In this case, it is desirable to suppress the LBE result, so that the surface is not highlighted to the user.

In step 710, the image processor calculates a neighbourhood surface score based upon the spatial variance of the target superpixel's 3D point descriptor from the obtained neighbourhood surface model.

The effect that the spatial variance has on the neighbourhood surface score may be configurable via a variance threshold configuration parameter which indicates a spatial variance from the surface which is considered to indicate object saliency. In one example, the image processor determines the neighbourhood surface score via a function that provides a result of 0 when the spatial variance is below the variance threshold configuration parameter, and provides a result that curves up to 1 sharply as the spatial variance exceeds the variance threshold configuration parameter.

FIG. 5b illustrates the neighbourhood surface score calculated by the image processor for the superpixels of the image 502, based on the superpixels' variances from the neighbourhood surface model of the image 502. As noted above, in the example illustrated in FIGS. 5a and 5b, the neighbourhood is the entire image 502, and the neighbourhood surface model is representative of the top surface 106 of the table 104.

In the example illustrated in FIGS. 5a and 5b, the image processor is further configured to differentiate between positive and negative spatial variance in the depth dimension z, such that superpixels which are located at a spatial variance deeper than the surface model have a lower neighbourhood surface score than superpixels which are located at the same spatial variance from the surface model but are at a closer depth.

Accordingly, the image processor has determined a high negative spatial variance for distance surfaces 112, thus the image processor has determined a neighbourhood surface score close to 0 for these surfaces 112. Accordingly, the LBE result for these distant surfaces 112 will be significantly suppressed and these surfaces will not be indicated as salient to the user in the artificial vision stimulus.

The image processor has determined a neighbourhood surface score of close to 0 for table surface 106, thus the LBE result for superpixels representing table surface 106 will be significantly suppressed and the table top 106 will not be indicated as salient to the user in the artificial vision stimulus.

Finally, the image processor has determined a neighbourhood surface score of close to 1 for the superpixels representing object 110. Accordingly, the LBE result for the superpixels representing object 110 will be preserved and the saliency of this object will be indicated to the user via the artificial vision stimulus.

### Adjusting the LBE Result

In step 612, the image processor 202 adjusts the LBE result for a superpixel by incorporating the neighbourhood surface score for that superpixel. In one example, the LBE result and the neighbourhood surface score are combined through an unweighted multiplication. In other examples, weighted or conditional multiplication or summation methods may be used to combine the LBE result with the neighbourhood surface score to produce an adjusted LBE result for a superpixel.

### Repeat for Each Superpixel

The image processor repeats steps 604 to 612 for each superpixel in the list of selected superpixels as provided in step 304, in order to determine an adjusted LBE result for each of the selected superpixels.

### Determine Phosphene Values

Following the determination of the adjusted LBE result for each of the selected superpixels, the image processor 102 determines a phosphene value for each of the phosphene locations in the array of phosphene locations. For phosphene locations that are collocated with one of the selected superpixels, the phosphene value is determined to be the adjusted LBE result of that superpixel. For phosphene locations that are collocated with a non-selected superpixel, the phosphene value is determined to be zero.

The array of phosphene values represent salient object information regarding the field of view captured by the image and depth sensors. This salient object information may be visualised, by the visual stimulation device user, as an array of light intensities representing the form and location of salient objects.

### Post-Processing

Optionally, and depending on example requirements and operational parameters, the image processor may perform post-processing on the array of phosphene values to improve the effectiveness of the salient object information.

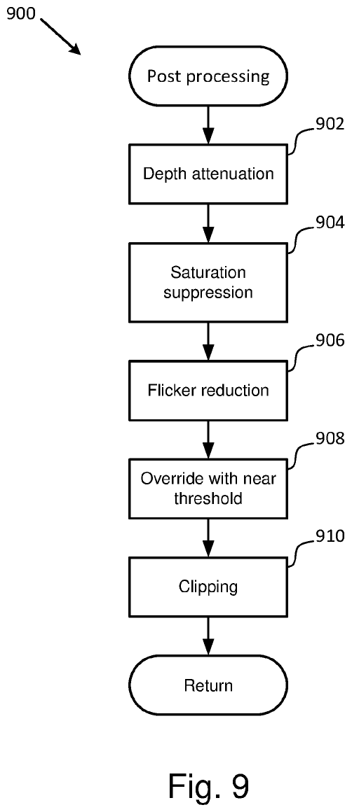

An example embodiment of the post-processing method is illustrated in FIG. 9. It is to be understood that method 900 is a non-limiting example of post-processing steps that an image processor may perform following the determination of the phosphene value for phosphene locations. In some embodiments, the image processor 202 performs all of steps 902 to 912, in the order shown in FIG. 9. Alternatively, the image processor may perform only a subset of steps 902 to 912 and/or perform the steps of method 900 in an alternative order to that shown FIG. 9.

### Perform Depth Attenuation

In step 902, the image processor 202 may dampen each phosphene value in accordance with a depth attenuation configuration parameter. For example, the method may calculate a scaling factor according to: scale=1−(current_phosphene_depth*(1−depth_attenuation_percent))/max_distance which is then applied to the current phosphene value.

The depth attenuation adjustment results in nearer objects being brighter and farther objects being dimmer. For example, if the depth_attenuation_percent is set to 50%, and max_distance was 4.0 m, a phosphene value that was representing distance of 4.0 m would be dimmed by 50%, and one at 2.0 m would be dimmed by 25%.

### Perform Saturation Suppression

In step 904, the image processor may perform saturation suppression by calculating the global phosphene saturation by taking the mean value of all phosphene values. If the mean value is greater than a defined saturation threshold configuration parameter, the image processor performs a normalisation on the image to reduce the value of some phosphene values and thereby remove some saturation. Removing saturation of the phosphene values has the effect of drawing out detail within the visual stimulus.

### Flicker Reduction

The image processor may also be configured to perform the step of flicker reduction 906. Flicker reduction is a temporal feature to improve image stability and mitigate noise from both the depth camera data and adjusted LBE result. A flicker delta configuration parameter constrains the maximum amount a phosphene value can differ from one frame to the next, this is implemented simply by looking to the data from the last frame and making sure phosphene values do not change by more than this amount. Flicker reduction aims to mitigate flashing noise and enhances smooth changes in phosphene brightness.

Additionally, the image processor may be configured to set a phosphene value to 1 in the situation that a phosphene's depth value is closer than minimum depth. Furthermore, the image processor may be configured to clip or adjust phosphene values to accommodate input parameter restrictions of the implanted visual stimulation device.

### Generate Visual Stimulus

Once the image processor has calculated the adjusted LBE result for each of the selected superpixels, determined the phosphene value for each of the phosphene locations and has performed any post-processing functions configured for a specific embodiment, the image processor generates the visual stimulus. The image processor 202 then communicates the visual stimulus to the visual stimulation device 212, via output 221.

The visual stimulus may be in the form of a list phosphene values, with one phosphene value for each phosphene location on the grid of phosphene locations. In another example, the visual stimulus may comprise differential values, indicating the difference in value for each phosphene location, compared to the corresponding phosphene value at the previous image frame. In other examples, the visual stimulus is a signal for each electrode and may include an intensity for each electrode, such as stimulation current, or may comprise the actual stimulation pulses, where the pulse width defines the stimulation intensity.

In one example, the phosphene locations correspond with the spatially arranged implanted electrodes 214, such that the low resolution image formed by the grid of phosphenes may be reproduced as real phosphenes within the visual cortex of the user. Real phosphenes is the name given to the perceptual artefact caused by electrical stimulation on an electrically stimulating visual prosthetic.

In one example, a simulated phosphene display may be used to validate the method described herein and may consist of a 35×30 rectangular grid scaled to image size. Each phosphene has a circular Gaussian profile whose centre value and standard deviation is modulated by brightness at that point. In addition, phosphenes sum their values when they overlap. In one example, phosphene rendering is performed at 8 bits of dynamic range per phosphene, which is an idealised representation. In a different example, it is assumed that maximum neuronal discrimination of electrical stimulation is closer to a 3 bit rendering. In another example, there are different numbers of bits of representation at each phosphene, and this may change over time.

In response to receiving the visual stimulus output from the image processor 202, the implanted visual stimulation device 212 stimulates the retina via the electrodes 214 at intensities corresponding with the values provided for each electrode. The electrodes 214 stimulate the visual cortex of the vision impaired user 211, triggering the generation of real phosphene artefacts at intensities broadly corresponding with the stimulation values. These real phosphenes provide the user with artificial vision of salient objects with the field of view 204 of the sensors 207.

In one example, the method 300, and associated sub-methods 400, 600, 700, 800 and 900, are applied to frames of video data, and the image processor generates visual stimulus on a per frame basis, to be applied to the electrodes periodically.

In one example, the visual stimulus is further adjusted to suit the needs of a particular vision impaired user or the characteristics of the vision impairment of that user. Furthermore, the adjustment of the visual stimulus may change over time due to factors such as polarisation of neurons.

In one example, the image processor adapts to the perception of the user on a frame by frame basis, where the visual stimulus is adjusted based on aspects of the user, such as the direction of gaze of the user's eyes.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### REFERENCES

[1] Local background enclosure for RGB-D salient object detection, Feng D, Barnes N, You S, et al., Proceedings of the IEEE Conference on Computer Vision and Pattern Recognition, 2016, 2343-2350.
[2] SLIC superpixels compared to state-of-the-art superpixel methods, Achanta R, Shaji A, Smith K, et al., PAMI, 34(11):2274-2282, 2012.

1. A method for creating artificial vision with an implantable visual stimulation device, the method comprising:
receiving image data comprising, for each of multiple points of the image, a depth value;
performing a local background enclosure calculation on the input image to determine salient object information; and
generating a visual stimulus to visualise the salient object information using the visual stimulation device,
wherein determining the salient object information is based on a spatial variance of at least one of the multiple points of the image in relation to a surface model that defines a surface in the input image.

2. The method of claim 1, wherein the surface model is a neighbourhood surface model which is spatially associated with the at least one of the multiple points of the image.

3. The method of claim 2, wherein determining the salient object information comprises determining a neighbourhood surface score for the at least one of the multiple points of the image, and wherein the neighbourhood surface score is based on a degree of the spatial variance of the at least one of the multiple points of the image from the neighbourhood surface model.

4. The method of claim 3, wherein the local background enclosure calculation comprises calculating a local background enclosure result for the at least one of the multiple points of the image.

5. The method of claim 4, wherein the method further comprises adjusting the local background enclosure result based on the neighbourhood surface score.

6. The method of claim 5, wherein adjusting the local background enclosure result comprises reducing the local background enclosure result based on the degree of spatial variance.

7. The method of claim 6, wherein the neighbourhood surface model is representative of a virtual surface defined by a plurality of points of the image in the neighbourhood of the at least one of the multiple points of the image.

8. The method of claim 2, further comprising spatially segmenting the image data into a plurality of superpixels, wherein each superpixel comprises one or more pixels of the image.

9. The method of claim 8, wherein the at least one of the multiple points of the image are contained in a selected superpixel of the plurality of superpixels.

10. The method of claim 9, wherein the neighbourhood comprises a plurality of neighbouring superpixels located adjacent to the selected superpixel.

11. The method of claim 9, wherein the neighbourhood comprises a plurality of neighbouring superpixels located within a radius around the selected superpixel.

12. The method of claim 9, wherein the neighbourhood comprises the entire image.

13. The method of claim 2, wherein the neighbourhood surface model is a planar surface model.

14. The method of claim 13, further comprising using a random sample consensus method to calculate the neighbourhood surface model for a target superpixel, based on a three dimensional location of the superpixels within the neighbourhood of the target superpixel.

15. The method of claim 1, wherein the method further comprises performing post-processing of the salient object information, and the post-processing comprises performing one or more of depth attenuation, saturation suppression and flicker reduction.

16. An artificial vision device for creating artificial vision, the artificial vision device comprising an image processor configured to:
receive image data comprising, for each of multiple points of the image, a depth value;
perform a local background enclosure calculation on the input image to determine salient object information; and
generate a visual stimulus to visualise the salient object information using the visual stimulation device,
wherein determining the salient object information is based on a spatial variance of at least one of the multiple points of the image in relation to a surface model that defines a surface in the input image.

17. The artificial vision device of claim 16, wherein the surface model is a neighbourhood surface model spatially associated with the at least one of the multiple points of the image.

18. The artificial vision device of claim 17, wherein determining the salient object information comprises determining a neighbourhood surface score for the at least one of the multiple points of the image, based on a degree of the spatial variance of the at least one of the multiple points of the image from the neighbourhood surface model.

19. The artificial vision device of claim 18, wherein the local background enclosure calculation comprises calculating a local background enclosure result for the at least one of the multiple points of the image.

20. The artificial vision device of claim 19, wherein the method further comprises adjusting the local background enclosure result based on the neighbourhood surface score.
